(19) United States
(12) Patent Application Publication
(10) Pub. No.: US 2013/0303940 A1
(43) Pub. Date: Nov. 14, 2013

(54) Audiometric Testing Devices and Methods

(71) Applicant: AUDIOLOGY INCORPORATED, Arden Hills, MN (US)

(72) Inventor: George L. Saly, Edina, MN (US)

(73) Assignee: Audiology Incorporated, Arden Hills, MN (US)

(21) Appl. No.: 13/891,511

(22) Filed: May 10, 2013

Related U.S. Application Data
(60) Provisional application No. 61/645,816, filed on May 11, 2012.

(51) Int. Cl. A61B 5/12

(52) U.S. Cl. CPC A61B 5/123
USPC 600/559

(57) ABSTRACT

Systems and methods for performing automated hearing tests using a hearing assistance device. A system may include a hearing assistance device and an interface device. The hearing assistance device may be configured to provide acoustic stimuli to a subject and the interface device may be configured to receive feedback from the subject in response to the acoustic stimuli. The system may generate results from the automated hearing test including hearing threshold levels. The results may be used to automatically adjust the hearing assistance device or may be automatically communicated. The results may also be communicated to a computing device of the system where a hearing professional may evaluate the results, recommend additional testing or manually adjust the hearing assistance device from the computing device.

### FIELD

This disclosure generally relates to devices, systems, and methods for testing auditory sensitivity.

### BACKGROUND

Hearing aids are programmed to provide appropriate amplification that properly accounts for the degree and configuration of a particular user's hearing loss. Hearing-impaired users of hearing aids may have fluctuating hearing loss or their hearing may change over time as a result of aging, sound exposure, and disease. A user's hearing loss can be tested periodically to learn of changes in the user's hearing loss. The test results can then be used to adjust the settings on the hearing aid appropriately to improve performance of the hearing aid. A common method of testing hearing is for a hearing aid user to visit a hearing professional (e.g., audiologist) to receive a hearing test with an audiometer.

### SUMMARY OF INVENTION

A method for testing the hearing of a subject is provided. The method includes providing a hearing assistance device including at least one programmable processer, enabling an interface device to communicate with the hearing assistance device and receive a response from the user, the interface device including at least one programmable processor, providing acoustic stimuli to the subject using the hearing assistance device, receiving from the subject, responses to the acoustic stimuli using the interface device, adaptively selecting acoustic stimuli to provide to the subject based on the subject's responses, and generating results based on the subject's responses.

The step of generating results may be accomplished by identifying hearing thresholds based on the subject's responses, and deriving quality indicators based upon the acoustic stimuli provided and the subject's responses, the quality indicators including at least one of false positive response probabilities, number of trials, time per trial, and test-retest differences. The method may also include the step of producing a diagnostic audiogram based on the results, the step of configuring the hearing assistance device based on the results, and the step of automatically communicating the results to a computing device.

A system for testing the hearing of a subject is provided. The system includes a hearing assistance device including at least one programmable processor, an interface device including at least one programmable processor, and one or more memory modules including executable instructions. The executable instructions may cause at least one programmable processor to provide acoustic stimuli to the subject using the hearing assistance device, receive from the subject responses to the acoustic stimuli using the interface device, adaptively selecting acoustic stimuli to provide to the subject based on the subject's responses, and generate results based on the subject's responses to the acoustic stimuli. The hearing assistance device and the interface device may be configured to communicate via a communication link.

In some examples, the one or more memory modules including the executable instructions may be included in the hearing assistance device, the interface device, or distributed between the two devices. The communication link may be a wired or wireless connection. In some examples, the system may include a computing device configured to communicate with the hearing assistance device and the interface device, the computing device including at least one programmable processor. The one or more memory modules including the executable instructions may be included in the computing device. The computing device may be configured to communicate with the hearing assistance device and the interface device via the communication link. In other examples, the computing device may be configured to communicate with the hearing assistance device and the interface device via a second communication link. The second communication link may be a wireless protocol with access to an internet connection. The system may also be configured to automatically communicate the results of the test to the computing device.

A non-transitory computer-readable storage article including executable instructions to cause at least one programmable processor provide acoustic stimuli to a subject using a hearing assistance device, receive from the subject responses to the acoustic stimuli using an interface device, adaptively selecting acoustic stimuli to provide to the subject based on the subject's responses, and generate results based on the subject's responses to the acoustic stimuli.

### BRIEF DESCRIPTION OF THE DRAWINGS

The following drawings illustrate particular examples and therefore do not limit the scope of the invention. The drawings are not to scale (unless so stated) and are intended for use in conjunction with the explanations in the following detailed description. Examples will hereinafter be described in conjunction with the appended drawings, wherein like numerals denote like elements.

FIG. 1A is a high-level schematic depiction of a system according to an example.

FIG. 1B is a high-level schematic depiction of a tone generator according to an example.

FIG. 2 is a flow diagram of a method for testing auditory sensitivity according to an example.

FIG. 3 is a high-level schematic depiction of a system according to an example.

FIG. 4 is an illustration of a trial structure according to an example.

FIG. 5 is a flow diagram illustrating logic for selecting test frequency and test ear for air-conduction testing according to an example.

FIG. 6 is a flow diagram illustrating a method for determining a threshold level according to an example.

### DETAILED DESCRIPTION

The following detailed description is exemplary in nature and is not intended to limit the scope, applicability, or configuration of the invention in any way. Rather, the following description provides some practical illustrations for implementing exemplary examples. Examples are provided for selected elements, and all other elements employ that which is known to those of ordinary skill in the field of the invention. Those skilled in the art will recognize that many of the noted examples have a variety of suitable alternatives.

Hearing assistance devices are often used to amplify sounds to assist the hearing of hearing-impaired individuals. Generally, hearing assistance devices must be programmed to provide the appropriate amplification to meet a particular individual's specific hearing needs. For example, a hearing test arrangement may be used to determine a patient's hearing level thresholds. A common method of testing hearing is for a patient to visit a hearing professional (e.g., audiologist). In some cases, the hearing professional may administer the hearing test manually or automatically with an audiometer. After testing the patient's hearing, the patient may then be fit with one or more hearing aids that are programmed using the level thresholds determined by the hearing test arrangement.

In many cases, the hearing of hearing-impaired individuals may fluctuate or change over time as a result of aging, disease, and/or sound exposure. In such cases, the hearing-impaired individual may periodically undergo additional hearing tests to identify changes in the user's hearing loss and to adjust the programming of the individual's hearing aids in light of the identified changes. The additional hearing tests may also be administered by a hearing professional manually or automatically with an audiometer.

According to some examples of the present application, an automated hearing test may be administered to an individual using a hearing assistance device instead of an audiometer. In some examples, the automated hearing test may be administered off-site without the assistance of a hearing professional. Thus a hearing-impaired individual may have a limited need, or no need, to visit a hearing professional or to use an audiometer. For example, one type of new hearing test arrangement may involve first fitting a patient with hearing aids based on general criteria provided by the patient (e.g., the patient's subjective reporting about their hearing difficulty or the patient's preferences). After fitting the hearing aids, an automated hearing test may be administered using the hearing aids and the results of the hearing test may then be used to automatically program the hearing aids. It is also contemplated that in some examples an initial hearing test and hearing aid programming may be conducted in the usual way by a hearing professional with an audiometer, and then subsequent automated hearing tests may be conducted with the hearing aids in place in the patient's ears to fine tune the programming of the hearing aids. Such examples may provide advantages over traditional hearing tests as automated hearing tests with the hearing aids in place may reduce, minimize, or eliminate sources of error associated with clinical hearing tests conducted with standard earphones. Further, administering an automated hearing test directly from the hearing aids without the use of an audiometer may provide cost benefits or less patient visits to a hearing professional. Other examples are contemplated wherein additional hearing tests may be administered remotely, but are directed and monitored by a hearing professional. A hearing professional may direct the automated hearing test by recommending a specific audiometric test (e.g., pure tone test or speech test) and then monitoring the results of the tests to ensure appropriate calibration of the hearing test or a change in condition of the patient.

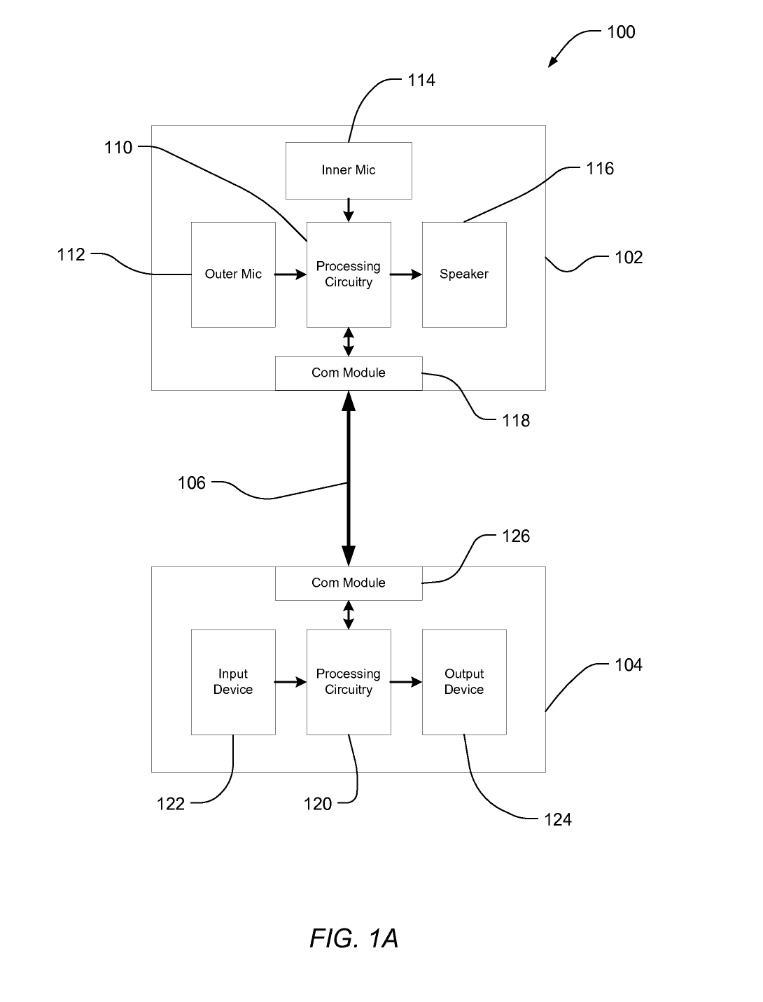

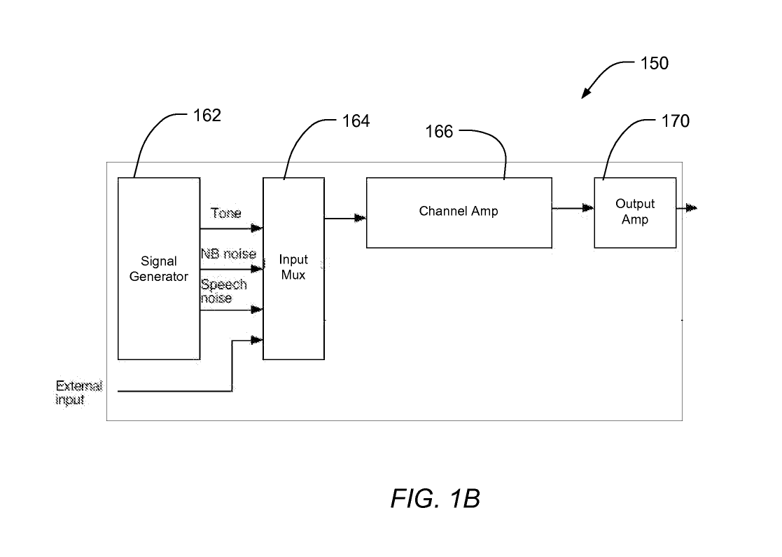

FIG. 1A is a high-level schematic depiction of a system 100 according to some examples of the invention. The system 100 includes a hearing assistance device 102 and an interface device 104. The hearing assistance device 102 may be a portable electronic device that can be worn by a person needing hearing assistance, for example a hearing aid. The hearing assistance device 102 may be in communication with interface device 104 through a communication link 106. In general, the interface device 104 may include a user interface (not shown) configured to receive inputs from the user and output data generated by the system 100 to a user.

According to some examples, the system 100 can be configured to perform an automated hearing test for a person or user wearing the hearing assistance device 102. In one example, the hearing assistance device 102 may be a hearing aid worn by a user. The hearing aid may generate audible tones as part of an automated hearing test and the user may, in response to the tones, interact with the interface device 104. Based on the user's responses, system 100 may generate results of the automated hearing test and output the results of the test to the user with the interface device 104. In some examples, system 100 may also communicate the results to one or more additional devices (e.g., a hearing professional's computer, server or database). In some examples the hearing test results may be used to manually and/or automatically adjust the settings of the hearing aid to adapt the performance of the hearing aid according to the just-determined auditory sensitivity of the user. In some cases this may include an initial programming of the hearing aid and/or an adjustment of the hearing aid as the user's hearing changes over time. One example is a proprietary system conceived by Applicant referred to as “AidTAS,” which describes a Hearing Aid System for Testing Auditory Sensitivity.

In some examples, as shown in FIG. 1A, hearing assistance device 102 may comprise processing circuitry 110, an outer microphone 112, an inner microphone 114, a speaker 116, and a communication module 118. Hearing assistance device 102 may be a hearing aid that can be worn behind the ear, in the ear, or in the ear canal. Outer microphone 112 may be configured to detect sound waves from the external environment, convert the sound waves to an electrical signal, and communicate the electrical signal to processing circuitry 110. Processing circuitry 110 may be configured to process the electrical signal by, for example, filtering and amplifying the signal. Processing circuitry may also be configured to provide analog and/or digital audio processing. The processed signal may then be communicated to speaker 116 where the processed signal may be converted to sound waves and directed into the ear of a person wearing the hearing assistance device 102.

The processing circuitry 110 may include a number of well-known components. In some examples the processing circuitry 110 may include one or more programmable processors and one or more memory modules. The one or more programmable processors may include one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” may generally refer to any of the foregoing logic circuitry, alone or in combination with other logic circuitry, or any other equivalent circuitry. In some examples, the processor(s) may contain instructions to perform one or more tasks.

According to some examples, instructions may also be stored in the memory module(s) for programming the processor(s) to perform one or more tasks or to store data generated or collected by the hearing assistance device. The one or more memory modules may include a non-transitory computer-readable medium, such as a computer-readable storage medium, containing instructions. Instructions embedded or encoded in a computer-readable storage medium may cause a programmable processor, or other processor, to perform the methods of the disclosure, e.g., when the instructions are executed. Non-transitory computer readable storage media may include volatile and/or non-volatile memory forms including, e.g., random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

In some examples, instructions stored in either the memory modules(s) or the programmable processor(s) may be modified or updated based on instructions received from system 100 via communication module 118. Those skilled in the art will appreciate that the teachings provided herein may be implemented in a number of different manners with, e.g., hardware, firmware, and/or software.

According to some examples, the hearing assistance device 102 may also include a tone generator that can be used to generate pure tones at various frequencies and intensities according to a desired hearing test scheme. In some cases, the tone generator may be part of the processing circuitry 110. In some cases, the tone generator may be considered to be separate from the processing circuitry 110. In some cases the tone generator may be provided by circuit components such as processors, amplifiers, and the like that are included in known types of hearing assistance devices.

FIG. 1B illustrates one example of a tone generator 150 that allows a hearing assistance device such as the device 102 in FIG. 1A to generate pure tones at various frequencies and intensities according to instructions received from the processing circuitry 110. In some cases the tone generator 150 may also generate masking noise for masking an ear not being tested and/or speech noise. For example, in some cases the tone generator 150 may generate narrow band (NB) noise and/or speech noise for use during an audiometric test.

In general, the tone generator 150 includes a signal generator 162, such as a tunable oscillator that is capable of generating signals having a range of frequencies. The signal generator 162 is coupled with an input multiplexer 164 that routes one or more distinct inputs into a channel amplifier 166. For example, the input multiplexer 164 may receive several inputs, such as a pure tone, narrow band noise, speech noise, and one or more external inputs. In some examples the external inputs are provided by processing circuitry (e.g., processing circuitry 110 in FIG. 1A) and/or may be generated based on inputs received from an interface device (e.g., interface device 104 in FIG. 1A).

The channel amplifier 166 may be coupled to an output amplifier 170, which can vary the intensity level of a signal to a desired testing level (e.g., as instructed by processing circuitry 110). Although not shown, in practice the output amplifier is directly or indirectly coupled with a transducer of the hearing assistance device, such as the speaker 116 shown in FIG. 1A. Pure tones and/or other sounds are then converted by the transducer to, e.g., sound pressures, for audiometric testing with the hearing assistance device.

Returning to FIG. 1A, hearing assistance device 102 may also include an inner microphone 114. The inner microphone 114 can be used in conjunction with the processing circuitry 110 to calibrate, or otherwise adjust, operation of the hearing assistance device 102 (e.g., by adjusting the processing circuitry and/or a tone generator) based on the actual output of the speaker 116 and/or the physical properties of the user's ear that receives the output. For example, in some cases the hearing assistance device 102 can be thought of as being uncalibrated in the same sense that a clinical audiometer may be uncalibrated. In one calibration scheme, the inner microphone 114 can be used to measure the levels of hearing test signals in the ear canal, which enables measurement of the transduced signals in terms of physical sound intensity units. The measured sound pressure levels can then be compared to the desired signal levels and the hearing assistance device 102 can be calibrated by adjusting operation based on the differences between the measured sound pressure levels and the desired sound pressure levels.

Some examples of methods of calibration that may be used for calibrating the hearing assistance device 102 are described in U.S. Patent Application 2011/0009770, to Margolis et al., titled Audiometric Testing and Calibration Devices and Methods, the content of which is hereby incorporated herein by reference in its entirety. In some cases, operation of the hearing assistance device 102 may be adjusted using programming software loaded into the processing circuitry 110 by the hearing aid's manufacturer. In some cases, aftermarket software and methods of calibration may be uploaded to the processing circuitry and used to calibrate operation of the hearing assistance device.

Of course, it should be appreciated that the depiction of the hearing assistance device 102 is a highly simplified, high-level diagram for purposes of the present disclosure, and those skilled in the art will understand that the hearing assistance device 102 may include a wide variety of components implemented in hardware, software and/or firmware. In addition, the hearing assistance device 102 may provide many different functionalities depending upon the design of the particular hearing assistance device 102. As just one example, the hearing assistance device 102 may be configured to provide one or more hearing assistance functions that may or may not be included in existing devices such as hearing aids, and may also provide pure tone generation, hearing sensitivity testing, and operational adjustment routines based on the testing results. In some examples the hearing assistance device 102 is an analog-digital or completely digital hearing aid that can be worn behind the ear, in the ear, or in the ear canal. For example, the hearing aid may provide analog and/or digital audio processing and include a programmable control circuit that expands the functionality of the hearing aid.

As noted above, system 100 may also include an interface device 104. Interface device may include, among other things, an input device 122, an output device 124, processing circuitry 120, and communication module 126. According to some examples, interface device 104 may be a personal computing device such as a desktop PC, a laptop, a tablet computer, a personal digital assistant, a cell phone or smart phone, or any other type of computing device.

In some examples, input device 122 may be configured to receive inputs from the user (e.g., feedback and/or responses to the automated hearing test). Output device 122 may be configured to provide instructions or inform the Input device 122 and output device 124 may be any suitable input/output technology, including devices that provide physical, aural, or other types of interfaces for the user to interact with the interface device 104. In some examples, interface device 104 may be a computer or laptop wherein input device 122 includes a keyboard and/or mouse and output device 124 includes an electronic display. In certain examples, a single device may be configured to provide the functionality of both input device 122 and output device 124. For example, interface device 104 may be a smartphone or tablet including a touch screen that may be used to receive input from the user and output information to the user. In examples where the automated test administered is a speech test, input device 122 may be voice/speech recognition technology. Further, input device 122 and output device 124 need not be integrated into interface device 104, as in the case where interface device 104 is a computer with peripherals including a keyboard, a mouse, and an electronic display.

Interface device 104 may also include processing circuitry 120 configured to provide certain functionality for the interface device 104. Processing circuitry may be provided in any suitable form and may include a number of well-known components. In some examples the processing circuitry 120 includes one or more programmable processors and one or more memory modules. Instructions can be stored in the memory module(s) for programming the processor(s) to perform one or more tasks. In alternate examples, the processor(s) may contain instructions to perform one or more tasks, such as, for example, in cases where a field programmable gate array (FPGA) or application specific integrated circuit (ASIC) are used. The processing circuitry (e.g., processor) is not limited to any specific configuration. In some examples, instructions stored in either the memory modules(s) or the programmable processor(s) may be modified or updated based on instructions received from system 100 via communication module 126. Those skilled in the art will appreciate that the teachings provided herein may be implemented in a number of different manners with, e.g., hardware, firmware, and/or software.

As noted above, both interface device 104 and hearing assistance device 102 may each include a communication module. The communication modules may be configured to enable inter-device communication between the hearing assistance device and the interface device over the communication link 106. As will be appreciated, any suitable communication technology may be utilized depending upon the available types of communication links and other design factors. In one example, communication link 106 may be provided by a cable (e.g., serial, USB, microUSB, etc.) and the communication modules 118 and 126 may include the appropriate cable jacks for the cable. In another example, communication link 106 may be a wireless link (e.g., 802.11b/g/n, Bluetooth) and the communication modules 118 and 126 may include a wireless transceiver for sending and receiving wireless transmission over the wireless link. In some examples, the communication modules of the hearing assistance device and/or the interface device may be configured to be turned off or placed into a sleep mode during non-testing periods to conserve energy of the devices.

Inter-device communication need not be exclusive between hearing assistance device 102 and interface device 104. For example, hearing assistance device 102 may be configured to use communication link 106 to communicate with more than one interface device, and conversely, interface device 104 may be configured to communicate with more than one hearing assistance device. As will be discussed further below, according to some examples hearing assistance device 102 and interface device 104 may be configured to communicate with other types of devices. For example, system 100 may include additional devices, and hearing assistance device 102 and interface device 104 may be configured to communicate with the additional devices using communication link 106. Further, communication between hearing assistance device 102 and interface device 104 need not be direct, rather communication may be conveyed via intermediary devices. In one example, communication link 106 may be a wireless link using 802.11 technology wherein hearing assistance device 102 communicates with interface device 104, or an additional device, via a wireless access point connected to a local area network and/or the internet.

According to some examples, hearing assistance device 102 and/or interface device 104 may each include more than one communication module. As can be appreciated, different communication technologies may be suited for varying ranges of communication. For example a communication link utilizing a cable may be preferred for short distances due to its reliability and speed, while a wireless communication link may be preferred for long range communication as it may utilize one or more wireless networks (e.g., a mobile telephone network or a wireless local area network connected to the internet, etc.). As will be discussed further herein, certain examples may include a hearing assistance device and/or an interface device with more than one communication module.

As mentioned above, in some examples the system 100 is configured to perform one or more automated hearing tests for a user wearing the hearing assistance device 102. In some cases the hearing assistance device 102 generates audible tones as part of a hearing test that are directed into one of the user's ears due to the placement of the hearing assistance device 102 proximate the ear. Upon hearing one or more tones, the user may respond to the tones by interacting with the interface device 104. The system 100 can then determine the results of the hearing test, which may then be stored, output, and/or used to adjust the settings of the hearing assistance device 102 to provide an improved performance for the user.

The system 100 can be configured to execute an automated hearing test in a number of different ways. According to some examples, the hearing assistance device 102 is configured to administer the automated hearing test and the interface device is simply used to enable interaction with the hearing assistance device 102. For example, processing circuitry 110 of the hearing assistance device 102 may be configured to execute and control the automated hearing test via software instructions programmed in memory and executed by a programmable processor. In one example, processing circuitry 110 may be configured to instruct a tone generator within the hearing assistance device to produce a series of tones according to a hearing test protocol, which are then delivered to the user's ear with the speaker 116. In this example, processing circuitry 120 of interface device 104 may be configured (e.g., via software instructions programmed into memory and executed by a processor) to display instructions associated with the automated hearing test to the user and receive inputs from the user (e.g., whether the user heard a tone generated by the hearing assistance device). The interface device 104 can then communicate the inputs received from the user to the processing circuitry 110 in the hearing assistance device 102, which controls the test. Processing circuitry 110 of the hearing assistance device may optionally adjust the settings of the hearing assistance device 102 according to the test results.

According to some examples, system 100 may be configured such that interface device 104 administers the automated hearing test instead of hearing assistance device 102. Interface device 104 may be configured to execute and control the hearing test as well as receive inputs from the user and hearing assistance device 102 may be configured only to generate tones during the automated hearing test. In some examples, the processing circuitry 120 of the interface device 104 may be configured to execute and control the hearing test via software instructions programmed in memory and executed by a programmable computer processor. In this case, the processing circuitry 120 of the interface device 104 may instruct, via communication link 106, a tone generator within the hearing assistance device 102 to produce a series of tones according to a hearing test protocol, which are then delivered to the user's ear with the speaker 116. The processing circuitry 120 of interface device 104 may also be configured (e.g., via software instructions programmed into memory and executed by a processor) to display instructions associated with the automated hearing test to the user and receive feedback from the user. The interface device 104 can then use the inputs received from the user to determine the results of the hearing test and may optionally adjust the settings of the hearing assistance device 102 according to the test results.

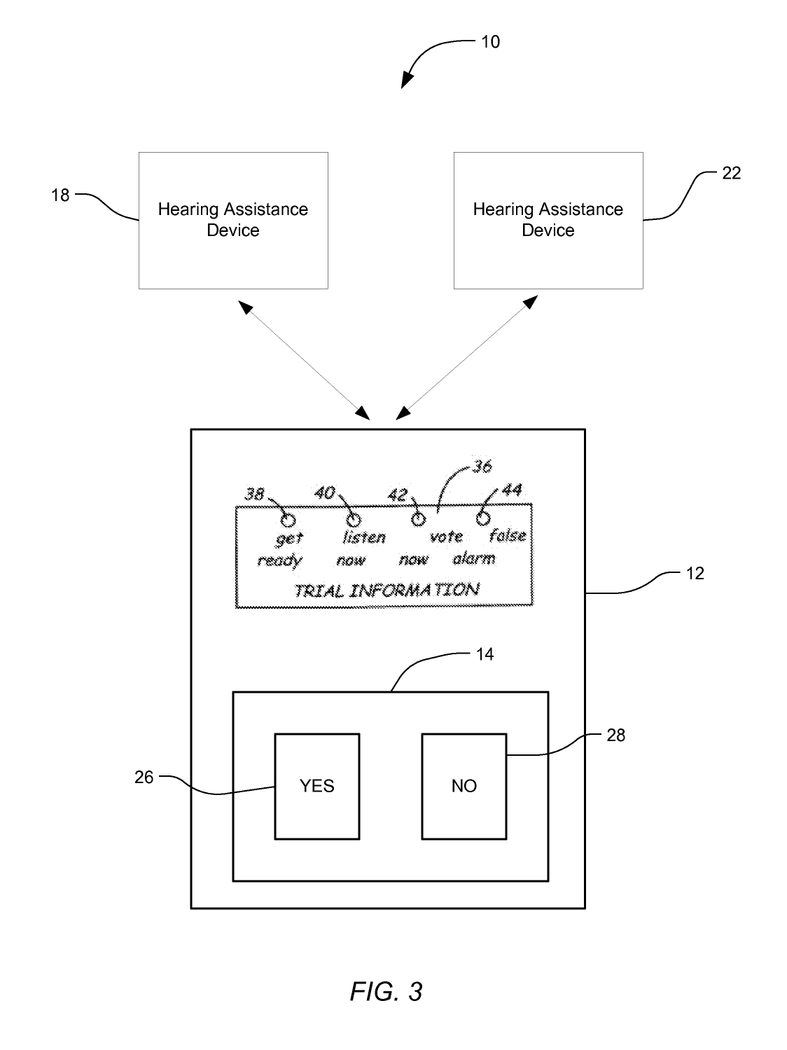

According to some examples, a hearing test may be administered by portions of processing circuitry in both the hearing assistance device 102 and the interface device 104. Accordingly, examples are not limited to a particular control configuration, but may be implemented with a variety of localized processing circuitry (e.g., mostly or completely within one device) or distributed processing circuitry (split among multiple devices). As noted above, in some examples a system may include more than one hearing assistance device 102 and/or more than one interface device 104. For example, as will be discussed with reference to FIG. 3, in some cases a system may include two hearing assistance devices (e.g., a left hearing aid and a right hearing aid) that communicate with a common interface device (e.g., a smart phone). It should be appreciated that a wide variety of configurations of hearing assistance devices and/or interface devices are possible and examples are not limited to any specific configuration.

According to some examples, system 100 may include additional devices that may be configured to help administer automated hearing tests via a hearing assistance device. Additional devices may include, but are not limited to, a computer in a doctor's office or a server. In one example, an automated hearing test may be administered via a hearing assistance device in a hearing professional's office and the results of the test may be communicated by the hearing assistance device and/or the interface device via a communication link to a computer in the doctor's office wherein the test results may be stored as a part of the patient's medical records and/or to be reviewed by the hearing professional. This configuration of system 100 provides the advantages of allowing a hearing professional to passively or actively monitor a patient's hearing condition based on the results of the test and recommend appropriate treatment as needed, and/or verify that the automated hearing tests were properly administered based on the results of the test.

In some examples, the automated hearing test may be administered by an additional device. For example, the hearing assistance device may be used only to generate the appropriate tones for the automated hearing test and the interface device may be used only to receive and communicate feedback from the user. The additional device may execute and control the automated hearing test via a communication link by directing the hearing assistance device to generate a series of tones according to a hearing test protocol. Further, the additional device may be configured to receive the user's response inputted into the interface device. In some examples, the additional device may be a computing device in a hearing professional's office or a server. The additional device may include software instructions programmed in memory of the additional device and executed by one or more programmable processors of the additional device. In one example, a hearing professional may use the additional device to direct a patient to take an automated hearing test and may further direct a type of hearing test that should be taken by the patient. For example, a hearing professional may use a computer and direct a patient that it is time for a hearing checkup by using the computer to communicate with a smartphone belonging to the patient. When the patient is ready, the computer may administer an automated hearing test by directing the hearing assistance device to generate a series of tones according to a hearing test protocol and receive from the interface device a response of the patient to the tones. In examples where the automated hearing test is administered by the hearing assistance device and/or interface device, the computer may be configured to allow the hearing professional to “authorize” a hearing test. After the automated hearing test is administered, the computer generates the results which may be stored and/or reviewed by the hearing professional. In some examples, the computer may be configured to allow the hearing professional to administer additional or different audiometric tests either initially or based on the results of the initial test. As can be appreciated, and as noted above, the additional device may be in the same or different geographical location as the hearing assistance device and the interface device and therefore the hearing assistance device and the interface device may include more than one communication module as appropriate to communicate with all the devices of system 100.

The examples above are provided not as limitations to system 100, but rather to demonstrate that many different configurations of the system exist and may be used as appropriate for different applications of an automated hearing test administered via a hearing assistance device. One skilled in the art will appreciate the advantages of system 100 and how it may be configured and adapted to streamline and make reliable a variety of aspects of audiometric testing.

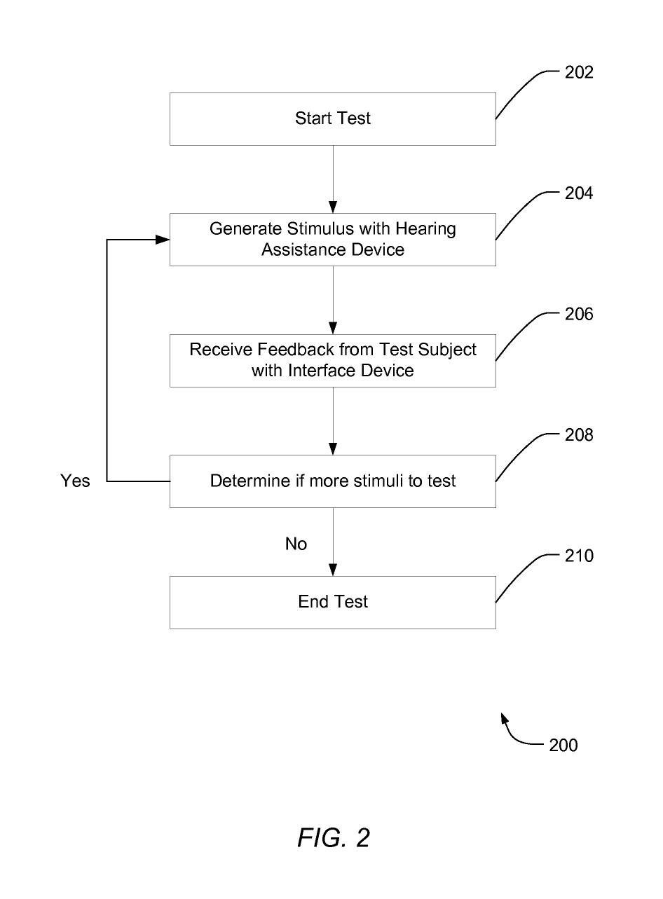

FIG. 2 is a flow diagram of a method 200 for automated testing of auditory sensitivity according to an example. According to some examples, the method 200 includes starting an automated hearing test 202 with a hearing assistance device test system, such as the system 100 illustrated in FIG. 1A. As part of the hearing test, a stimulus (such as an audible tone) may be generated by the hearing assistance device 204 and directed into a user's ear. The user may then indicate whether he or she heard or did not hear the stimulus. The interface device receives 206 this feedback from the user. The system may then store, process, or otherwise handle the user input and then determine 208 if another stimulus should be tested. If yes, the method returns to generate additional stimuli and receive feedback from the user. If no, the test ends 210.

Of course the method 200 depicted in FIG. 2 is just one example of a hearing test according to one example and the system may execute methods for testing hearing that include a variety of steps. Accordingly, the method 200 could include more or less steps depending upon the particular hearing test being executed. As just a few examples, methods for testing auditory sensitivity may include pure tone audiometry wherein one, two, or more pure tones are generated in order to test a user's hearing and/or may include additional steps for adjusting operation of a hearing assistance device based on test results. In some cases, methods may include speech audiometry wherein auditory sensitivity may be tested by generating speech stimuli (e.g., predetermined words at particular volumes) with a hearing assistance device and then determining (e.g., via a microphone and processing circuitry) whether the subject is able to repeat the generated speech. In such examples, an input device of the user interface may include speech recognition technology to capture the user's response. In other examples, methods may include Bekesy audiometry wherein the user interacts with the interface device to trace monoaural thresholds for pure tones. According to other examples, methods for testing auditory sensitivity may include both bone conducted acoustic stimuli and air conducted acoustic stimuli. It can be appreciated that a system 100 may be configured to automatically perform one or more types of hearing tests.

According to some examples, a system such as the system 100 illustrated in FIG. 1A is configured to implement a validated pure-tone hearing test that may produce results comparable to results obtained by a licensed audiologist using a traditional audiometer. Examples of such a test is described in U.S. Pat. No. 6,496,585, filed Jan. 27, 2000, and titled “Adaptive apparatus and method for testing auditory sensitivity.” The entire content of U.S. Pat. No. 6,496,585 is hereby incorporated herein by reference in its entirety. Further examples of hearing tests that may be implemented with a system including a hearing assistance device are described in U.S. Pat. No. 7,704,216, filed Aug. 24, 2005 and titled “Method for Assessing the Accuracy of Test Results.” The entire content of U.S. Pat. No. 7,704,216 is hereby incorporated herein by reference in its entirety. As noted above, however, the types of automated audiometric testing administered by system 100 are not limited to the above references.

The following is a discussion of how the adaptive hearing tests described in U.S. Pat. No. 6,496,585, and/or variations of the tests, may be implemented with systems described herein, such as systems including a hearing assistance device and an interface device similar to the system 100 shown in FIG. 1A.

Table 1 lists some definitions of terms and symbols used in the following disclosure.

TABLE 1
Definition of Terms

| Term (Default Value) | Symbol | Definition |
|---|---|---|
| Subject | S | The person being tested |
| Examiner | E | The person administering the test |
| Trial | | A sequence of temporal intervals corresponding to one stimulus |
| | | presentation |
| Ready Interval | Iᵣ | The first temporal interval of a trial; the interval preceding the |
| | | stimulus |
| Observation Interval | Iₒ | The temporal interval following the Ready Interval; the interval |
| | | in which the stimulus is presented; Iₒ has a duration dₒ |
| Vote Interval | Iᵥ | The temporal interval following the Observation Interval; Iᵥ |
| | | begins at the offset of Iₒ and ends when the subject responds |
| Level | L | The level of a stimulus; for auditory stimuli L may be specified |
| | | sound pressure level or hearing level |
| Initial Level (40 dB HL) | Lᵢ | L of the first stimulus presentation in a threshold determination |
| Initial Increment (10 dB) | ΔLᵢ | The amount that the level is incremented when a “No” response |
| | | occurs to the initial level |
| Stimulus Decrement (10 dB) | ΔL_{d} | The amount that the level is decremented when a “Yes” |
| | | response occurs |
| Stimulus Increment (5 dB) | ΔL″ | The amount that the level is incremented following “No” |
| | | responses that occur after the first “Yes” response |
| Maximum Level | Lₘ | Maximum value of a level for a specified stimulus |
| Criterion Level | L_{c} | The level corresponding to a “Yes” response immediately |
| | | preceded by a “No” response. |
| Threshold Criterion | C | Number of times the criterion level must occur at a given level |
| | | to meet the definition of a threshold level |
| Threshold Level | Lₜ | Level corresponding to threshold; level at which the criterion |
| | | level occurs at the threshold criterion |
| Number of Stimuli | Nₛ | Number of stimulus presentations required to determine a |
| | | threshold level |
| Masking Criterion | M | In the masking mode, the minimum level for which masking is |
| | | presented to the non-test ear |
| Interaural Attenuation | IA | The estimated difference in stimulus level in the test ear and |
| | | non-test ear |
| Masker Level | ML | The level of the masking noise presented to the non-test ear |
| Masker Level at Threshold | MLₜ | The level of the masking noise presented to the non-test ear |
| | | when the test signal level is a threshold level |
| Test-Retest Difference at 1 kHz | ΔT₁ₖ or | Difference threshold level for two 1 kHz or 0.5 kHz threshold |
| or 0.5 kHz | ΔT_{0.5k} | measures |
| Catch Trial | | A trial for which the observation interval contains no stimulus |
| Catch Trial Probability (20%) | P_{c} | The probability that a trial will be a catch trial |
| False Response Probability | P_{y} | Proportion of “Yes” responses in catch trials; determined for |
| | | each test stimulus |
| Feedback | | Information provided to subject indicating that a “Yes” vote |
| | | occurred during a catch trial |
| Octave Threshold Difference | D | Difference between adjacent octave frequencies above which |
| Criterion | | the interoctave frequency is tested |
| Time per Trial | T | The total duration of the test divided by the number of trials |

FIG. 3 is an example of a system 10 that provides an Adaptive Method for Testing Auditory Sensitivity (AMTAS) according to some examples. System 10 includes an interface device 12 in the form of a smart phone wirelessly connected to a first hearing assistance device 18 and a second hearing assistance device 22, which in this example are configured as hearing aids. System 10 may be configured to provide wireless communication through any suitable wireless communication protocol over any suitable frequency spectrum. Examples of potentially useful wireless communication may occur over radio frequencies in an open and/or closed network such as a Wi-Fi network, a mobile phone network, and/or dedicated or proprietary band of frequencies. Interface device 12 includes an input section 14 that provides a yes button 26 and no button 28. In the case of a smart phone, the input section 14 can be a portion of a touch-sensitive screen and the yes and no buttons 26, 28 may be computer generated images displayed on the screen. The interface device 12 also includes an output section 36, which may optionally be provided by a touchscreen in the case of a smartphone. Output section 36 further includes a get ready indicator 38, a listen now indicator 40, a vote now indicator 42, and a false alarm indicator 44, which may be computer generated graphics displayed on a touchscreen in the case of a smartphone.

According to one example, in operation the system 10 presents instructions to a user, also referred to herein as a subject (S) as follows: You are going to hear some tones. Most of them will be very soft. The tone may be in either ear. When the tone occurs it will always be while the “Listen Now” indicator is on. When the “Vote Now” indicator comes on, I want you to tell me if you think there was a tone when the “Listen Now” indicator was on. Push the YES button if you think there was a tone. Push the NO button if you did not hear a tone. You must push the YES button or the NO button when the “Vote Now” indicator comes on. The “False Alarm” indicator will come on if you pushed the YES button when there was no tone. You may hear some noise that sounds like static. If you hear a noise, ignore it and only push the YES button if you hear a tone.

According to the illustrated example, the user or subject places hearing aids 18 and 22 on or behind his or her ears. In some cases the user may only use one hearing aid (or other hearing assistance device) to test a single ear at one time. In some cases two hearing aids (or other hearing assistance devices) may be used, one for each ear, and masking noise, though not always required, may optionally be presented to the ear not currently being tested. Processing circuitry installed in the system 10 (e.g., running software instructions within the smart phone and/or within one or more of the hearing aids) carries out S's hearing test automatically.

Threshold levels, Lₜ, are determined for a set of air conducted auditory stimuli specified by the system 10. Stimuli are pure tones of varying frequency. In some cases test frequencies are selected from those listed in Table 2. Frequencies shown in italics are default test frequencies in some cases.

TABLE 2
Test Frequencies (kHz)

| | | | | | | | | | | | |
|---|---|---|---|---|---|---|---|---|---|---|---|
| Air | 0.125 | 0.25 | 0.5 | 0.75 | 1.0 | 1.5 | 2.0 | 3.0 | 4.0 | 6.0 | 8.0 |

In some cases the system 10 may use the default set of stimuli or another set of stimuli selected from the frequencies in Table 2. The default set includes audiometric frequencies that are required for a diagnostic hearing evaluation and additional frequencies are automatically tested when needed.

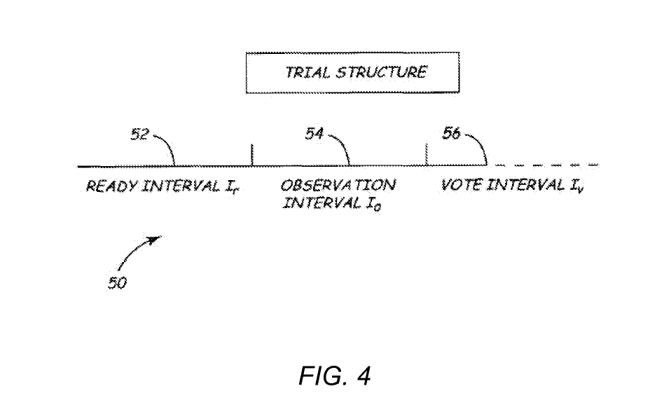

Each stimulus is presented in a trial, which is illustrated in FIG. 4. Trial structure 50 consists of Ready Interval (Iᵣ) 52 of duration dᵣ, Observation Interval (Iₒ) 54 of duration dₒ, followed by Vote Interval (Iᵥ) 56 of variable duration.

The testing is performed using a psychophysical method, which is an adaptive Yes/No procedure. The stimulus is presented during Iₒ 54. S responds during Iᵥ 56 by pushing Yes Button 26 if a stimulus was detected during Iₒ 54 or No Button 28 if no stimulus was detected in Iₒ 54. Iᵥ 56 ends when S responds. Catch trials, trials in which no stimulus is presented in Iₒ, are performed randomly with a predetermined probability, P_{c}, to determine S's reliability. Feedback is used to inform S when a “Yes” response occurred during a catch trial. False Alarm indicator 44 lights when S presses Yes button 26 during each catch trial.

In some cases the rate of stimulus presentation is determined by S's response time, allowing S to control the pace of the test. This permits testing of subjects with a wide range of age, cognitive ability, reaction time, and motor dexterity. Trials are presented repetitively at various stimulus levels L until Lₜ is determined. The process is repeated for all specified stimuli or the default stimulus set.

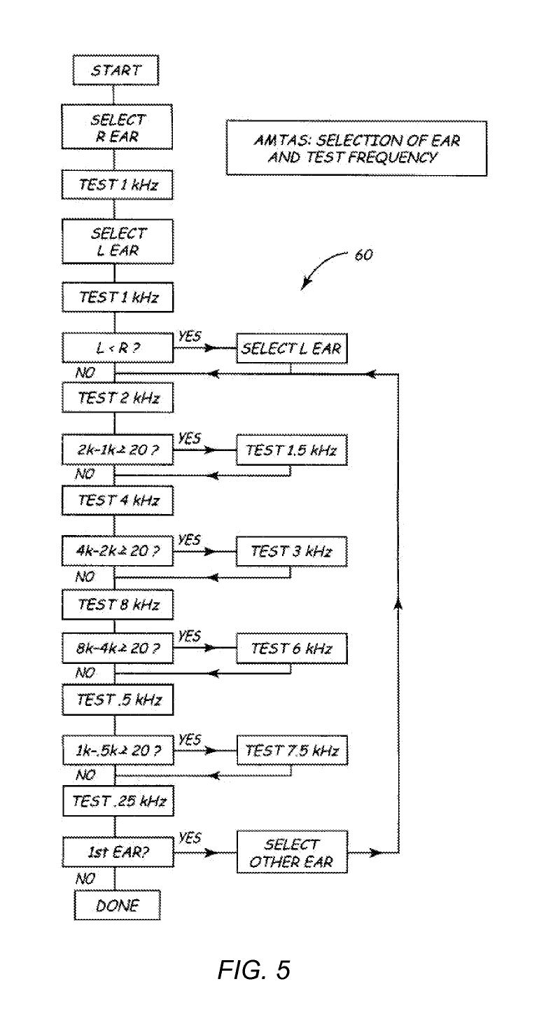

FIG. 5, consisting of flowchart 60, illustrates the logic for the selection of test frequency and test ear for air-conduction testing using the default stimulus set according to some examples. The default initial test ear for air-conduction testing in this case is the right ear. Lₜ at 1 kHz is determined for the right ear and then for the left ear. The test ear for subsequent stimuli is the ear with the better Lₜ at 1 kHz. For air-conduction testing, the default order of test frequencies is the following: 1 kHz, 2 kHz, 4 kHz, 8 kHz, 0.5 kHz, and 0.25 kHz. Interoctave frequencies (0.75 kHz, 1.5 kHz, 3 kHz, and 6 kHz) are automatically tested when the difference between two adjacent octave frequencies exceeds D, where D is a predetermined value. The default value of D is 20 decibels (dB). After Lₜ is determined for all frequencies, the test is repeated at 1 kHz unless Lₜ>Lₘ, where Lₘ is the maximum value of L for a specified stimulus, in which case 0.5 kHz is retested. The difference in the two 1 kHz thresholds, ΔT_{1 k} (or 0.5 kHz, ΔT_{0.5 k}), is a measure of test reliability. After thresholds are tested for each selected frequency, the other ear is tested.

In some cases, when the test signal may be audible in the non-test ear, a masking signal is automatically presented to ensure that perception of the test signal by the non-test ear does not affect the test. For example, when testing with air-conducted stimuli, masking may optionally be presented to the non-test ear in Iₒ when L>M, where M is the masking criterion. M is the level at which the stimulus may be audible in the non-test ear of a normal hearing subject for a given stimulus/transducer combination. The masking level, ML (in effective masking level), presented to the contralateral ear is L-IA+10 dB where IA is the average interaural attenuation. M and IA are dependent on the stimulus and the hearing aid transducer.

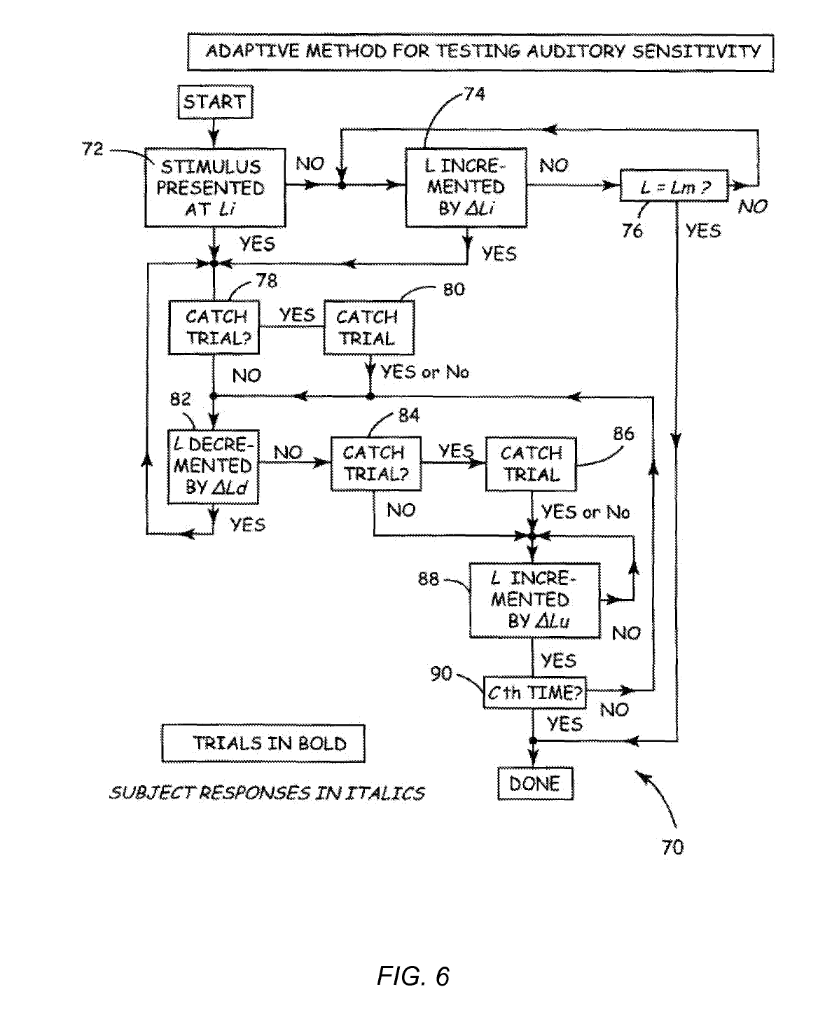

FIG. 6 illustrates an example of the steps in determining Lₜ by adaptively varying L. Lₜ is the lowest level at which S hears a tone at least 50% of the time. Adaptive method 70 of FIG. 46 includes Initial step 72, Increment step 74, Maximum Threshold step 76, Catch trials 78 and 80, Decrement step 82, Catch trials 84 and 86, Increment step 88, and C Value step 90.

In operation, the initial stimulus, Lᵢ, is presented to S at Initial step 72. If S responds “No” to Lᵢ, L of the next stimulus is presented at L+ΔLᵢ at Increment step 74. Increment step 74 is repeated by incrementing L by ΔLᵢ until a “Yes” response occurs or until L=Lₘ. If L reaches Lₘ then Lₜ>Lₘ.

If S responds “Yes” to Lᵢ, Catch trial 78 is performed to provide an indication of S's reliability. If S responds “Yes” to Catch trial 78, then False Alarm indicator 44 illuminates and Catch trial 80 is performed. Regardless of S's response to Catch trial 80, testing continues. If, however, S responds “No” to Catch trial 78, testing continues without performing Catch trial 80.

When testing continues, L of the next stimulus is presented at L-ΔL_{d} at Decrement step 82. After each “Yes” response, Catch trials 78 and 80 are performed again, and L is subsequently decremented by ΔL_{d}. If S responds “No” at Decrement step 82, Catch trials 84 and 86 are performed as described above for Catch trials 78 and 80. For each “No” response after the first “Yes” response at Decrement step 82, L is incremented by ΔLᵤ, which is shown at Increment step 88.

L that produces a “Yes” response immediately preceded by a “No” response is designated L_{c}. When L_{c} occurs C times at the same value of L, where C is the threshold criterion, that level is designated Lₜ. This is illustrated by C Value step 90. In some cases the default value of C is 2, but C can set to be any value.

The number of stimulus presentations, Nₛ, required to determine Lₜ is a quality indicator. Adaptive method 70 is repeated for each selected stimulus or for the default stimulus set.

The proportion of “Yes” votes following Catch trials 78, 80, 84, and 86, designated P_{y}, is a measure of response reliability. P_{y} is determined for each Lₜ and an average P_{y} is reported for each ear and for both ears combined.

In some examples, results are presented in standard audiogram format. The quality indicators listed in Table 4 can be reported in some cases.

TABLE 4
Quality Indicators
QUALITY INDICATORS

| | |
|---|---|
| P_{y} (f) | False alarm probability at each test frequency |
| P_{y} (ear) | False alarm probability for ear. |
| P_{y} (S) | False alarm probability for both ears combined |
| Nₜ (f) | Number of trials required to determine Lₜ for each |
| | frequency |
| ΔT₁ₖ or ΔT_{0.5k} | Test-retest difference at 1 kHz or 0.5 kHz |
| T | Time per trial |

In some cases, for each threshold measurement, two quality indicators are reported, P_{y} and Nₜ. In addition, P_{y} is reported for each ear and both ears combined. ΔT_{1 k} or ΔT_{0.5 k} is also reported. Values of each quality indicator that exceed two standard deviations beyond the mean are identified. MLₜ, the masker level at threshold, is reported for each threshold and Masking Alerts are identified.

According to some examples, system 10 and the corresponding method for adaptively testing auditory sensitivity select a test ear and test frequency, provide contralateral masking when appropriate, and quantitatively assess test reliability. In some examples, a system and the corresponding method it implements are designed to eliminate the major sources of human error that influence the accuracy of manual pure tone audiometry. A summary of some possible features of systems and methods in some examples contrasted with manual pure tone audiometry are presented in Table 5.

TABLE 5

| Manual Pure Tone | Some Examples Providing Systems |
|---|---|
| Audiometry | and Methods for Testing Hearing |
| Requires continuous control by E | No intervention by E required |
| E selects test ear and test | Test ear and test frequencies |
| frequencies | automatically selected to produce |
| Provides only qualitative | complete diagnostic audiogram |
| assessment of test reliability | provides five quantitative, |
| which is highly dependent on E's | E-independent measures of test |
| experience | reliability |
| Requires E to determine the need | Automatically presents appropriate |
| for masking the non-test ear and | masking noise to non-test ear |
| to manually select masker levels | |
| Does not identify thresholds that | Alerts to thresholds that may be |
| are likely to be inaccurate | inaccurate due to inappropriate |
| | masking or subject inconsistency |

Thus, some examples of the invention are disclosed. Although some examples have been described in considerable detail, the disclosed examples are presented for purposes of illustration and not limitation and other examples of the invention are possible. One skilled in the art will appreciate that various changes, adaptations, and modifications may be made without departing from the spirit of the invention and the scope of the disclosure.

What is claimed is:

1. A method for testing the hearing of a subject comprising:
providing a hearing assistance device including at least one programmable processor;
enabling an interface device to communicate with the hearing assistance device and receive a response from the subject, the interface device including at least one programmable processor;
providing acoustic stimuli to the subject using the hearing assistance device;
receiving, from the subject, responses to the acoustic stimuli using the interface device;
adaptively selecting acoustic stimuli to provide to the subject based on the subject's responses; and
generating results based on the subject's responses to the acoustic stimuli.

2. The method of claim 1, wherein the step of generating results comprises:
identifying hearing thresholds based on the subject's responses; and
deriving quality indicators based upon the acoustic stimuli provided and the subject's responses, the quality indicators including at least one of false response probabilities, number of trials, time per trial, and test-retest differences.

3. The method of claim 1, further comprising the step of producing a diagnostic audiogram based on the results.

4. The method of claim 1, further comprising the step of automatically configuring the hearing assistance device based on the results.

5. The method of claim 1, further comprising the step of automatically communicating the results to a computing device.

6. A system for testing the hearing of a subject comprising:
a hearing assistance device including at least one programmable processor;
an interface device including at least one programmable processor;
one or more memory modules including executable instructions to cause at least one programmable processor to:
provide acoustic stimuli to the subject using the hearing assistance device;
receive, from the subject, responses to the acoustic stimuli using the interface device;
adaptively selecting acoustic stimuli to provide to the subject based on the subject's responses; and
generate results based on the subject's responses to the acoustic stimuli;
wherein the hearing assistance device and the interface device are configured to communicate via a communication link.

7. The system of claim 6, wherein the one or more memory modules including the executable instructions is included in the hearing assistance device.

8. The system of claim 6, wherein the one or more memory modules including the executable instructions is included in the interface device.

9. The system of claim 6, wherein the one or more memory modules including the executable instructions is distributed between the hearing assistance device and the interface device.

10. The system of claim 6, wherein the communication link is a wired connection.

11. The system of claim 6, wherein the communication link is a wireless connection.

12. The system of claim 6, wherein the one or more memory modules including the executable instructions further causes the at least one programmable processor to:
identify hearing thresholds based on the subject's responses; and
derive quality indicators based upon the acoustic stimuli provided and the subject's responses, the quality indicators including at least one of false response probabilities, number of trials, time per trial, and test-retest differences.

13. The system of claim 6, wherein the one or more memory modules including the executable instructions further causes the at least one programmable processor to produce a diagnostic audiogram based on the results.

14. The system of claim 6, wherein the one or more memory modules including the executable instructions further causes the at least one programmable processor to automatically configure the hearing assistance device based on the results.

15. The system of claim 6, further comprising a computing device configured to communicate with the hearing assistance device and the interface device, the computing device including at least one programmable processor.

16. The system of claim 15, wherein the one or more memory modules including the executable instructions is included in the computing device.

17. The system of claim 15, wherein the hearing assistance device and the interface device are configured to communicate with the computing device via the communication link.

18. The system of claim 15, wherein the hearing assistance device and the interface device are configured to communicate with the computing device via a second communication link.

19. The system of claim 18, wherein the second communication link is a wireless protocol with access to an internet connection.

20. The system of claim 15, wherein the one or more memory modules including the executable instructions further causes the at least one programmable processor to automatically communicate the results to the computing device.

21. A non-transitory computer-readable storage article having executable instructions stored thereon to cause at least one programmable processor to:
provide acoustic stimuli to a subject using a hearing assistance device;
receive, from the subject, responses to the acoustic stimuli using an interface device;
adaptively selecting acoustic stimuli to provide to the subject based on the subject's responses; and
generate results based on the subject's responses to the acoustic stimuli.

22. The article of claim 21, further comprising executable instructions to cause the at least one processor to:
identify hearing thresholds based on the subject's responses; and
derive quality indicators based upon the acoustic stimuli provided and the subject's responses, the quality indicators including at least one of false response probabilities, number of trials and test-retest differences.

23. The article of claim 21, further comprising executable instructions to cause the at least one processor to produce a diagnostic audiogram based on the results.

24. The article of claim 21, further comprising executable instructions to cause the at least one processor to automatically configure the hearing assistance device based on the results.

25. The article of claim 21, wherein the acoustic stimuli may be bone conducted stimuli.

26. The article of claim 21, further comprising executable instructions to cause the at least one processor to automatically communicate the results to a computing device.
